(19) United States
(12) Patent Application Publication
(10) Pub. No.: US 2021/0186903 A1
(43) Pub. Date: Jun. 24, 2021

(54) TREATMENT FOR DEMYELINATING DISEASE

(71) Applicant: CAMBRIDGE ENTERPRISE LIMITED, Cambridge, Cambridgeshire (GB)

(72) Inventors: Robin FRANKLIN, Cambridge (GB); Björn NEUMANN, Cambridge (GB); Peter VAN WIJNGAARDEN, East Melbourne (AU)

(73) Assignee: CAMBRIDGE ENTERPRISE LIMITED, Cambridge, Cambridgeshire (GB)

(21) Appl. No.: 17/050,543

(22) PCT Filed: Apr. 26, 2018
(86) PCT No.: PCT/EP2018/060774

§ 371 Date: Oct. 26, 2020

(51) Int. Cl. A61K 31/155; A61K 31/192; A61K 31/40; A61K 31/439; A61K 31/4174; A61K 31/198

(52) U.S. Cl. CPC A61K 31/155; A61K 31/192; A61K 31/198; A61K 31/439; A61K 31/4174; A61K 31/40

(57) ABSTRACT

This invention relates to the use of AMPK agonists, such as metformin to, restore the responsiveness of aged oligodendrocyte progenitor cells (OPCs) to differentiation factors. This may be useful in promoting oligodendrocyte differentiation and increasing the remyelination of neuronal axons, for example in the treatment of demyelinating diseases, such as multiple sclerosis (MS). Therapeutic combinations comprising AMPK agonist and a differentiation factor and therapeutic uses thereof are provided.

### FIELD

The present invention relates to methods and compounds for promoting the remyelination of neuronal axons, for example in the treatment of demyelinating diseases, such as multiple sclerosis.

### BACKGROUND

The ability of the adult to regenerate oligodendrocytes, the myelin forming cell of the central nervous system (CNS), contrasts with the poor capacity to regenerate neurons in most brain regions¹. Oligodendrocyte progenitor cells (OPCs) are responsible for oligodendrocyte formation throughout life and for remyelination in the setting of white matter injury^{2,3}. Remyelination has been studied in a wide range of experimental animal models, and, as with most regenerative processes, the efficiency of remyelination declines progressively with ageing^{4,5}.

In aged individuals, remyelination can become so slow that it effectively fails. This has important implications for chronic demyelinating diseases, such as multiple sclerosis (MS), which can be of several decades' duration. Delayed remyelination renders denuded axons susceptible to irreversible degeneration, a phenomenon that underpins the progressive neurological decline associated with the later stages of MS⁶. The mechanisms that regulate OPC differentiation are dysregulated in the ageing brain⁷ due to age-related changes in the cells and molecules within the environment in which remyelination occurs^{8,9}. These changes can be overcome, in principle, by providing a more youthful systemic environment that is permissive for regeneration¹⁰. Thus, remyelination can potentially be enhanced by pro-differentiation factors lacking in the aged brain. However, it remains unclear whether OPCs undergo intrinsic changes with ageing that affect their responsiveness to differentiation signals.

### SUMMARY

The present inventors have unexpectedly discovered that AMPK agonists restore the responsiveness of aged oligodendrocyte progenitor cells (OPCs) to differentiation factors. This may be useful in increasing the production of oligodendrocytes and promoting the remyelination of neuronal axons, for example in the treatment of demyelinating diseases, such as multiple sclerosis (MS).

A first aspect of the invention provides a therapeutic combination comprising an AMPK agonist and a differentiation factor.

A second aspect of the invention provides a method of treating a demyelinating disease comprising administering to an individual in need thereof a therapeutic combination according to the first aspect.

A third aspect of the invention provides a therapeutic combination of the first aspect for use in a method of treating a demyelinating disease, for example a method of the second aspect.

A fourth aspect of the invention provides an AMPK agonist for use in a method of treating a demyelinating disease comprising administering said AMPK agonist in combination with a differentiation factor to an individual in need thereof, for example a method of the second aspect.

A fifth aspect of the invention provides a differentiation factor for use in a method of treating a demyelinating disease comprising administering said differentiation factor in combination with an AMPK agonist to an individual in need thereof, for example a method of the second aspect.

The demyelinating disease of the first to fifth aspects may be multiple sclerosis (MS).

Suitable AMPK agonists for use in the first to fifth aspects may include metformin.

Suitable differentiation factors include for use in the first to fifth aspects may include clemastine, benzatropine, miconazole, bexarotene and thyroid hormone.

Other aspects and embodiments of the invention are described in more detail below.

### BRIEF DESCRIPTION OF THE FIGURES

FIG. 1 shows OPCs lose their inherent capacity for differentiation with ageing. FIG. 1A shows representative images of young adult (2-3 months old) and aged OPCs (20-24 months old) differentiated in the absence of growth factor or in the presence of T3. Increasing maturity is visualized using O4 (early), CNPase (intermediate), MBP (mature) marker of the oligodendrocyte lineage. Scale bars: 50 μm. FIG. 1B shows quantification of O4+Olig2+ cells during the time course. FIG. 1C shows quantification data for CNPase+Olig2+ cells. FIG. 1D shows quantification of MBP+Olig2+ cells. Statistical significance was determined using One-Way ANOVA for each timepoint and Dunnett's multiple comparison test of each sample against “Aged-T3”. All data are presented as mean±SD (n=3). Statistical significance was determined using One-Way ANOVA for each timepoint and Dunnett's multiple comparison test of each sample against “Aged-T3”. FIG. 1E shows quantification of morphological differentiation. CNP/MBP+ cells were scored as complex when they had at least 4 main branches with several sub-branches as shown in (F). All data are presented as mean±SD (n=3 biological replicates for each 268 age group, One Way ANOVA with Dunnett's multiple comparison test of each sample against “Aged-T3”). *P<0.05, **P<0.01, ***P<0.001.

FIG. 2 shows aged OPCs have reduced expression of OPC specific genes and acquire hallmarks of ageing. FIG. 2A shows a volcanoplot visualizing RNAseq data from young adult (2-3 months old) and aged OPCs (20-24 months old). Among the genes significantly higher expressed in young adult OPCs (1.5 fold change, p.adj.<0.05, magenta dots) are typical 7 OPC genes such as Pdgfra, Ascl1 and Sox6. Genes significantly higher expressed in aged OPCs (1.5 fold change, p.adj<0.05, blue dots) contain differentiation markers such as Enpp6 and Cnp1. FIG. 2B shows OPC specific genes tested for differential expression between young and aged OPCs. The pie chart summarises the findings as the percentage of genes that were significantly higher expressed in aged or young OPCs (p.adj.<0.05) or that were not differentially expressed (p>0.05). FIG. 2C shows qRT-PCR for selected genes to confirm the RNAseq comparing freshly isolated young and aged OPCs (n=3 biological replicates for each age group, two-tailed t-test). FIG. 2D shows top 5 pathways identified by Ingenuity pathway analysis (z-score >2 and p.adj.<0.05) for genes enriched in aged OPCs (p.adj<0.05). FIG. 2E shows representative images for comet assays (alkaline conditions) of freshly isolated young and aged OPCs to visualize the degree of DNA damage. FIG. 2F shows quantification of the comet assay. The categories used for scoring are depicted in the respective boxes. Statistical significance was determined using two tailed t-test for each damage category. All data are presented as mean±SD (n=3 biological replicates for each age group). FIG. 2G shows qRT-PCR results visualizing expression of the senescence marker Cdkn2a. Data is presented as mean±SD (n=3 biological replicates for each age group, two-tailed t-test). *P<0.05, **P<0.01, ***P<0.001.

FIG. 3 shows that ADF enhances remyelination in aged rats partially through the restoration of OPC differentiation capacity. FIG. 3A shows a schematic of the fasting experiment. ADF animals have access to food only on alternating days. Control animals had free access. Fasting was started at 12 months of age for 6 months. Animals were lesioned in the white matter by focal injection of ethidium bromide (EB) into the caudal cerebellar peduncule (CCP). FIG. 3B shows remyelination was assessed 50 days later in semi-thin resin sections stained with toluidine blue. Remyelinated axons appear as circles with a distinct grey border. Myelinated axons that have not undergone demyelination are surrounded by thick black myelin. Demyelinated axons appear as feint circles with feint border. Scale bars: 100 μm. FIG. 3C shows electron micrographs from areas within the lesion center. Scale bars: 5 μm. FIG. 3D shows quantification of the remyelination data. Each dot represents one animal. The rank corresponds to the degree of remyelination, whereby a higher rank indicates better remyelination (n≥6 for each group, Mann-Whitney-U test). FIG. 3E shows representative images visualizing differentiating oligodendrocytes (Olig2+CC1+ cells) within the lesion center at 50 days post lesion (dpi). FIG. 3F shows quantification of the density of newly formed oligodendrocytes within the lesion at 21 dpl and 50 dpl. FIG. 3G shows quantification of the proportion of newly formed oligodendrocytes among all lineage cells (Olig2+) (n=4 biological replicates for each group in E+F, two-tailed t-test). FIG. 3H shows differentiation assay of OPCs that were isolated from 18 month old animals that underwent fasting or had free access to food as described in 3A. Differentiated oligodendrocytes were visualized at day 10 of differentiation as MBP+Olig2+ cells. Scale bars: 50 μm. FIG. 3I shows quantification of the proportion of differentiated cells among all lineage cells. FIG. 3J shows quantification of the scoring for morphological differentiation criteria as shown in FIG. 1. (n=3 biological replicates for each group for 1+J, two-tailed t-test). *P<0.05, **P<0.01, ***P<0.001, ns P>0.05.

FIG. 4 shows that metformin ameliorates hallmarks of ageing and restores the ability of aged OPCs to differentiate. FIG. 4A shows OPCs isolated from aged animals (≥18 months) and cultured in the presence of growth factors for 5 days. Some cells were treated with 100 μM metformin with each medium change during the first 5 days (days 2 and 4). FIG. 4B shows qRT-PCR for OPC genes and FIG. 4C for Cdkn2a. All data are presented as mean±SD (n=3, two329 tailed t-test). FIG. 4D shows quantification of comet assays. All data are presented as mean±SD (n=3, two-tailed t-test comparing each damage category between the two experimental groups). FIG. 4E shows representative images of differentiation assay data. Newly formed oligodendrocytes are identified as Olig2+MBP+ cells. Scale bars: 100 μm. FIG. 4F shows Quantification of the differentiation assay as the proportion of MBP among all lineage cells. All data are presented as mean±SD (n=3, One-Way ANOVA with Dunnett's multiple comparison test against “Aged noGF”). *P<0.05, 335 **P<0.01, ***P<0.001, ns P>0.05.

FIG. 5 shows metformin treatment enhances remyelination in aged rats. FIG. 5A shows 12 month old female SD rats were divided in three groups. The control and ADF group were treated as described in FIG. 3. Metformin animals had ad libitum access to food but received metformin at dose of 300 mg/kg bodyweight in their drinking water from the age of 15 months. At 18 months of age demyelinating lesions were induced by injection of ethidumbromide (EB) into the caudal cerebellar peduncule (CCP). FIG. 5B shows remyelination assessed 50 days later in semi-thin resin sections stained with toluidine blue. Remyelinated axons appear as circles with a distinct grey border. Not demyelinated axons are surrounded by thick black myelin. Demyelinated axons appear as feint circles with very feint border. Scale bars: 50 μm. FIG. 5C shows a ranking of the remyelination data. Each dot represents one animal. The rank corresponds to the degree of remyelination, whereby a higher rank indicates better remyelination (n=6 biological replicates for each group, Kruskal-Wallis test followed by Dunn's post test)*P<0.05

### DETAILED DESCRIPTION

This invention relates to treatment of demyelinating diseases, such as multiple sclerosis (MS), by administration of a differentiation factor in combination with an AMPK agonist. OPCs in an individual become progressively less sensitive to differentiation factors as the individual gets older, reducing the formation of differentiated oligodendrocytes that mediate remyelination. AMPK agonism is shown herein to restore the responsiveness of OPCs in the individual to differentiation factors, increasing the production of differentiated oligodendrocytes. This promotes remyelination and reduces or inhibits the degeneration of demyelinated neurons in the individual.

An AMPK agonist is a compound that promotes, enhances or increases the activity of the 5′ AMP-activated protein kinase (AMPK) pathway. Suitable methods for determining the activity of the AMPK pathway are well-known in the art (see for example Vincent et al (2015) Oncogene 34(28) 3627-3639). AMPK agonists are shown herein to increase the responsiveness of OPCs to differentiation factors. For example, a suitable AMPK agonist may augment the differentiation of aged (>12 months) rat OPCs in response to known inducers of differentiation in neonatal and young adult rat OPCs.

Suitable AMPK agonists include biguanides, such as metformin (N,N-dimethylbiguanide CAS: 657-24-9), buformin (1-butylbiguanide CAS: 692-13-7) and phenformin (phenethylbiguanide CAS: 114-86-3); AICAR (5-Aminoimidazole-4-carboxamide 1-β-D-ribofuranoside CAS: 3031-94-5); 2-deoxy-D-glucose (2DG; CAS 154-17-6); salicylate (2-Hydroxybenzoic acid; CAS 69-72-7); and A-769662 (6,7-dihydro-4-hydroxy-3-(2′-hydroxy[1,1′-biphenyl]-4-yl)-6-oxo-thieno[2,3-b] pyridine-5-carbonitrile CAS 844499-71-4).

AMPK agonists may be synthesised using conventional synthetic routes or obtained from commercial suppliers.

In some preferred embodiments, the AMPK agonist may be metformin.

The AMPK agonist is administered as described herein in combination with a differentiation factor. A differentiation factor is an agent that promotes or increases the differentiation of responsive oligodendrocyte progenitor cells (OPCs) into oligodendrocytes. This differentiation increases the number of oligodendrocytes in the vicinity of a demyelinated neuronal axon, thereby increasing remyelination and reducing degeneration of the demyelinated neuronal axon.

Suitable differentiation factors may for example increase the differentiation of neonatal or young adult rat (<12 months) OPCs into oligodendrocytes and may promote remyelination in neonatal or young adult rats in the absence of AMPK agonists.

Suitable differentiation factors include retinoid X receptor (RXR) agonists, such as bexarotene (CAS 153559-49-0); selective histamine Hi antagonists, such as clemastine (CAS 15686-51-8); selective M1 muscarinic acetylcholine receptor antagonists, such as benzatropine (CAS 86-13-5) and solifenacin (CAS 242478-37-1); glucocorticoid receptor agonists, such as clobestasol (CAS 25122-46-7), mitogen-activated protein kinase pathway agonists, such as miconazole (CAS 22916-47-8); K-opioid receptor agonists, such as U-50488 (CAS 67198-13-4); endothelin receptor pan-antagonists, such as PD142-893 (CAS 155893-16-6), EDNRB agonists, such as BQ3020 (CAS 143113-45-5); GPR17 antagonists, such as pranlukast (CAS 103177-37-3) and montelukast (CAS 158966-92-8); and thyroid hormone receptor agonists, such as thyroid hormone, e.g. triiodothyroxine (T3 (2S)-2-amino-3-[4-(4-hydroxy-3-iodophenoxy)-3,5-diiodophenyl]propanoic acid CAS 6893-02-3) and thyroxine (T4; (3,5,3′,5′-tetraiodothyronine CAS 51-48-9). Other suitable differentiation factors that promote oligodendrocyte differentiation are described in Cole et al Glia (2017) 65 1565-1589.

Therapeutic combinations of AMPK agonists and differentiation factors as described herein may be useful in methods of OPC differentiation and/or promoting remyelination in vitro or in vivo, for example in the treatment of demyelinating diseases in an individual.

AMPK agonists and differentiation factors will usually be administered to an individual in the form of pharmaceutical compositions, which may comprise at least one component in addition to the active agent.

In some embodiments, AMPK agonists and differentiation factors may be formulated into a single combined composition. For example, a pharmaceutical composition may comprise an AMPK agonist and a differentiation factor. In more preferred embodiments, the AMPK agonist and differentiation factor may be formulated into separate compositions. Separate compositions preparations may be useful, for example, to facilitate separate and sequential or simultaneous administration, and allow administration of the components by different routes or in varying relative doses.

A pharmaceutical composition may comprise, in addition to the AMPK agonist and/or differentiation factor, a pharmaceutically acceptable excipient, carrier, buffer, stabilizer or other materials well known to those skilled in the art. Such materials should be non-toxic and should not interfere with the efficacy of the active ingredient. The precise nature of the carrier or other material will depend on the route of administration. Suitable materials will be sterile and pyrogen free, with a suitable isotonicity and stability. Examples include sterile saline (e.g. 0.9% NaCl), water, dextrose, glycerol, ethanol or the like or combinations thereof. The composition may further contain auxiliary substances such as wetting agents, emulsifying agents, pH buffering agents or the like. Suitable carriers, excipients, etc. can be found in standard pharmaceutical texts, for example, Remington's Pharmaceutical Sciences, 18th edition, Mack Publishing Company, Easton, Pa., 1990.

The term “pharmaceutically acceptable” as used herein pertains to compounds, materials, compositions, and/or dosage forms which are, within the scope of sound medical judgement, suitable for use in contact with the tissues of a subject (e.g. pig or other mammal) without excessive toxicity, irritation, allergic response, or other problem or complication, commensurate with a reasonable benefit/risk ratio. Each carrier, excipient, etc. must also be “acceptable” in the sense of being compatible with the other ingredients of the composition.

Pharmaceutical compositions may be in the form of liquids, solutions, suspensions, emulsions, elixirs, syrups, tablets, lozenges, granules, powders, capsules, cachets, pills, ampoules, suppositories, pessaries, ointments, gels, pastes, creams, sprays, mists, foams, lotions, oils, boluses, electuaries, or aerosols.

Pharmaceutical compositions comprising the active compounds may be formulated in a dosage unit form that is appropriate for the intended route of administration.

The AMPK agonist and differentiation factor may be administered to a subject by any convenient route of administration, whether systemically/peripherally or at the site of desired action, including but not limited to, oral (e.g. by ingestion); and parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot, for example, subcutaneously or intramuscularly. Usually administration will be systemically, preferably orally, although other routes such as intraperitoneal, subcutaneous, transdermal, intravenous, nasal, intramuscular or other convenient routes are not excluded.

Pharmaceutical compositions may be prepared by any methods well-known in the art of pharmacy. Such methods include the step of bringing into association the active compound with the carrier which constitutes one or more accessory ingredients. In general, the compositions are prepared by uniformly and intimately bringing into association the active compound with liquid carriers or finely divided solid carriers or both, and then if necessary shaping the product.

Pharmaceutical compositions suitable for oral administration (e.g. by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste. A tablet may be made by conventional means, e.g., compression or moulding, optionally with one or more accessory ingredients. Compressed tablets may be prepared by compressing in a suitable machine the active compound in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolate, cross-linked povidone, cross-linked sodium carboxymethyl cellulose); surface-active or dispersing or wetting agents (e.g. sodium lauryl sulfate); and preservatives (e.g. methyl p-hydroxybenzoate, propyl p-hydroxybenzoate, ascorbic acid). Moulded tablets may be made by moulding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. For intravenous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringers Injection, Lactated Ringers Injection. Preservatives, stabilizers, buffers, antioxidants and/or other additives may be employed, as required. Many methods for the preparation of pharmaceutical formulations are known to those skilled in the art. See e.g. Robinson ed., Sustained and Controlled Release Drug Delivery Systems, Marcel Dekker, Inc., New York, 1978.

Optionally, other therapeutic agents may be included in the pharmaceutical composition.

Administration of the AMPK agonist and differentiation factor can include co-administration, either in a single pharmaceutical composition or using separate compositions, or consecutive administration in either order but generally within a time period such that both active agents can exert their biological activities simultaneously.

A therapeutic combination as described herein may be for use in a method of treatment of the human or animal body, for example a method of treating a demyelinating disease.

A demyelinating disease is a condition in which the myelin sheath which surrounds neurons in nervous tissue is lost or damaged, leading to axonal degeneration and impaired signal transduction in the affected nerves. Examples of demyelinating diseases include multiple sclerosis, transverse myelitis, optic neuritis, neuromyelitis optica, acute disseminated encephalomyelitis, idiopathic inflammatory demyelinating disease (IIDDs), central pontine myelinolysis, and progressive multifocal leukoencephalopathy. The demyelinating disease may be a chronic demyelinating disease.

In preferred embodiments, the demyelinating disease is multiple sclerosis (MS), most preferably progressive or neurodegenerative MS, for example primary or secondary progressive MS.

Individuals may be diagnosed with MS, progressive MS, or primary or secondary progressive MS in accordance with standard diagnostic criteria (McDonald W I, et al. Ann Neurol 2001; 50: 121-7; Fangerau T et al. Acta Neurol Scand 2004 109: 385-9).

In some embodiments, an individual may be non-responsive to the differentiation factor in the absence of the AMPK agonist. For example, treatment with the differentiation factor alone may not induce remyelination in the individual. This may occur for example when OPCs in an individual have become insensitive to differentiation factors through the aging process.

Suitable individuals may display no inflammation. For example, inflammation may have been previously suppressed in the individual.

Treatment may be any treatment or therapy, whether of a human or an animal, in which some desired therapeutic effect is achieved, for example, the inhibition or delay of the onset or progress of the demyelinating disease, and includes a reduction in the rate of progress, a halt in the rate of progress, amelioration of at least one symptom of the demyelinating disease, cure or remission (whether partial or total) of the demyelinating disease, preventing, delaying, abating or arresting one or more symptoms and/or signs of the demyelinating disease or prolonging survival of a subject or individual beyond that expected in the absence of treatment.

A therapeutic combination described herein may be administered to mammals, preferably humans. Administration may be in a “therapeutically effective amount”, this being sufficient to show benefit to a patient. The actual amount administered, and rate and time-course of administration, will depend on the nature and severity of what is being treated, the particular patient being treated, the clinical condition of the individual patient, the cause of the demyelinating disease, the site of delivery, the method of administration, the scheduling of administration and other factors known to medical practitioners. Prescription of treatment, e.g. decisions on dosage etc., is within the responsibility of general practitioners and other medical doctors, and may depend on the severity of the symptoms and/or progression of a disease being treated. Appropriate doses of AMPK agonists and differentiation factors are well known in the art. Specific dosages indicated herein, or in the Physician's Desk Reference (2003) as appropriate for the type of medicament being administered, may be used.

Treatments may be repeated at daily, twice-weekly, weekly or monthly intervals, at the discretion of the physician. Treatment may be periodic, and the period between administrations is about two weeks or more, e.g. about three weeks or more, about four weeks or more, or about once a month.

A therapeutic combination as described herein may be administered alone or in combination with other treatments, concurrently or sequentially or as a combined preparation with another therapeutic agent or agents, for the treatment of a demyelinating disease. For example, a therapeutic combination described herein may be used in combination with an existing therapeutic agent for demyelinating disease.

For example, in some embodiments, a therapeutic combination described herein may be used in combination with an anti-inflammatory or immunomodulatory compound, such as methyl-prednisolone, β-interferon, glatiramer acetate, teriflunomide, fingolimod, dimethyl fumarate (BG12), alemtuzumab, natalizumab or ocrelizumab.

Other aspects and embodiments of the invention provide the aspects and embodiments described above with the term “comprising” replaced by the term “consisting of” and the aspects and embodiments described above with the term “comprising” replaced by the term “consisting essentially of”.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

Modifications of the above embodiments, further embodiments and modifications thereof will be apparent to the skilled person on reading this disclosure, and as such, these are within the scope of the present invention.

All documents and sequence database entries mentioned in this specification are incorporated herein by reference in their entirety for all purposes.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

### EXPERIMENTAL

Materials and Methods

Animal Husbandry

All animal procedures were performed in compliance with United Kingdom Home Office regulations. The animals were housed under standard laboratory conditions on a 12 h light/dark cycle with constant access to food and water. All animals were housed in pairs or groups of up to 4 animals. For alternate day fasting (ADF) 12 months old female SD rats were restricted from food every other day. ADF animals had access to food on Tuesday, Thursday, Saturday and Sunday and all food was removed from their cages on Monday, Wednesday and Friday. The food was removed and returned in the mornings. The weight of each animal was weekly monitored. The fasting paradigm was interrupted for the first three days after surgery when all animals had free access to food. For metformin treatment 15 months old female SD rats that were fed ad libitum received metformin (Glucophage) in their drinking water (300 mg/kg bodyweight per day). Metformin treatment was interrupted for two days before and three days after surgery and then commenced to the end of the study (21 days after lesion induction). Fluid consumption was continuously monitored to adapt dosages.

Induction of White Matter Lesions and Assessment of Remyelination

For studies involving demyelination, female Sprague Dawley rats (Harlan Laboratories) 18 months of age were used. The rats were anesthetized with buprenorphine (0.03 mg/kg, s.c.) 471 and 2.5% isoflurane. Demyelination was induced by stereotaxic injection of 4 μl of 0.01% ethidium bromide (EB) into the caudal cerebellar peduncles (CCPs), as previously described 28. EB was delivered at a rate of 1 μl/min. After EB delivery the injection needle remained in position for additional 4 min.

To assess remyelination the rats were transcardially perfused with 4% glutaraldehyde and 0.4 mM CaCl₂) in PBS. The cerebellum was cut in to transverse 1 mm thick sections. The tissue was fixed in 2% osmium-tetroxide at 4° C. overnight, dehydrated through a series of washes in ethanol and propylene-oxide and embedded in resin. From the resin blocks 1 μm thick sections were cut and stained with 1% toluidine blue.

The lesions were ranked according to the degree of remyelination, whereby a higher rank was given to a sample with better remyelination. For electron microscopy (EM) ultrathin sections of lesion sites were cut and transferred onto copper grids. The sections were stained with uranyl acetate and imaging was performed using a Hitachi-H600 Transmission Electron Microscope.

Isolation of Adult Oligodendrocyte Progenitor Cells.

Adult rats (2-27 months) were decapitated after lethal injection of phenobarbital into the tail vein. The brains were removed quickly and placed into ice-cold isolation medium (Tab. S3, alternatively Hibernate A (Brainbits). The telencephalon and cerebellum were dissected in isolation medium; meninges, and the olfactory bulb were mechanically removed and the brain tissue was mechanically minced into 1 mm3 pieces. The tissue pieces were spun down at 100 g for 1 min at RT and the tissue was washed in HBSS− (no Mg2+ and Ca2+, Gibco). Each half of the brain was mixed with 5 ml of dissociation solution (34 U/ml papain (Worthington), 20 μg/ml DNAse Type IV (Gibco) in isolation medium). The brain tissue was dissociated on a shaker (50 rpm) 494 for 40 min at 35° C. The digestion was stopped by addition of ice cold HBSS−. The tissue was centrifuged (200 g, 3 min, RT), the supernatant completely aspirated and the tissue resuspended in isolation medium supplemented with 2% B27 and 2 mM sodium-pyruvate (trituration solution). The tissue was allowed to sit in this solution for 5 min. To obtain a single cell suspension the tissue suspension was triturated 10 times using first a 5 ml serological pipette and subsequently three fire polished glass pipettes (opening diameter >0.5 mm). After each trituration step the tissue suspension was allowed to sediment (approximately 1-2 min) and the supernatant (approximately 2 ml), containing the cells, was transferred into a fresh tube. After each round of trituration 2 ml of fresh trituration solution were added. To remove accidentally transferred undigested tissue bits, the collected supernatant was filtered through 70 μm cell strainers into tubes that contained 90% isotonic Percoll (GE Healthcare, 17-0891-01, in 10×PBS pH7.2 (Lifetech). The final volume was topped up with phenol-red free DMEM/F12 with HEPES (Gibco) and mixed to yield a homogenous suspension with a final Percoll concentration of 22.5%. The single cell suspension was separated from remaining debris particles by gradient density centrifugation (800 g, 20 min, RT, without break). The myelin debris and all layers without cells were discarded and the brain cell containing phase (last 2 ml) and cell pellet were resuspended in HBSS+ and combined in a fresh 15 ml tubes and centrifuged (300 g, 5 min, RT). The cell pellet was resuspended in red blood cell lysis buffer (Sigma, R7757) and incubated for 1 min at RT to remove red blood cells. 10 ml of HBSS+ were added to this cell suspension and spun down (300 g, 5 min, RT). The cell pellets were resuspended in 0.5 ml modified Milteny washing buffer (MWB, 2 mM EDTA, 2 mM Na-Pyruvate, 0.5% BSA in PBS, pH 7.3) supplemented with 10 ng/ml human recombinant insulin (Gibco). To this cell suspension 2.5 μg mouse-anti-rat-A2B5-IgM antibody (Millipore, Extended data Tab. 4) were added for every 10 million cells. After 25 min incubation, gently shaking at 4° C., 7 ml of 518 MWB were added. The solution was centrifuged (300 g, 5 min, RT) and the pellet resuspended in 80 μl MWB supplemented with 20 μl rat-anti-mouse-IgM antibody (Milteny, 130-047-302) per 10 million cells. The cells were incubated for 15 min, slowly shaking at 4° C. The secondary antibody was again washed out with 7 ml MWB and the sample was centrifuged (300 g, 5 min, RT). The cell pellet was resuspended in 0.5 ml and MACS was performed according to the recommendations of the supplier. Briefly, a MS column (Milteny, 130-042-201) were inserted into MiniMACS Separator (Miltenyi; 130-042-102) and pre-wet with 0.5 ml MWB. Resuspended cells were put onto one MS column. Subsequently the column was washed three times using 500 μl MWB for each wash. Finally A2B5 positive cells were flushed out the column with 1 ml pre-warmed, CO2 and O2 pre-equilibrated OPC medium.

Culture of Adult Oligodendrocyte Progenitor Cells.

Isolated OPCs were seeded onto 12 mm glass coverslips in 24 well plates (VWR) or into 96 well-plates (InVitro-Sciences) coated with PDL (Sigma). After isolation, OPCs were left to recover in OPC medium (60 μg/ml N-Acetyl cysteine (Sigma), 10 μg/ml human recombinant insulin (Gibco), 1 mM sodium pyruvate (Gibco), 50 μg/ml apo-transferrin (Sigma), 16.1 μg/ml putrescine (Sigma), 40 ng/ml sodium selenite (Sigma), 60 ng/ml progesterone (Sigma), 330 μg/ml bovine serum albumin (Sigma)) supplemented with b-FGF and PDGF (30 ng/ml each, Peprotech). OPCs were incubated at 37° C., 5% CO2 and 5% O2. The medium was completely exchanged to OPC medium with 20 ng/ml bFGF and PDGF after overnight culture to remove any dead cells. After 3 d the cell culture medium was switched to promote further proliferation (OPC medium+20 ng/ml bFGF and PDGF) or differentiation (OPCM+40 ng/ml T3). During differentiation or proliferation experiments 66% of the medium were replaced every 48 h and growth factors or other small molecules were added fresh to the culture. The culture medium used was 542 500 μl for cultures in 24 well plate wells and 150 μl for cultures in 96 well plate wells. For differentiation assays the medium was in some instances supplemented with 40 ng/ml thyroid-hormone (T3, Sigma), 50 nM 9-cis retinoic acid (9cRA, Sigma), 1 μM miconazole (Sigma, M3512) or 1.5 μM benztropine (Sigma, SML0847). Otherwise used small molecules: Rapamycin (Cell Guidance Systems, SM83-5) and metformin (Tocris, 2864).

Immunofluorescence for Tissue Sections

Rats were deeply anaesthetised by a lethal dose of pento-barbitol and transcardially perfused with 4% paraformaldehyde (PFA) in PBS. The brains were removed and post-fixed for 2 h at RT with 4% PFA. After a rinse in PBS the tissue was incubated in 20% sucrose solution (in PBS) overnight. The tissue was then imbedded in OCT-medium (TissueTek) and stored at −80° C. 12 μm sections were obtained using a cryostat. Tissue sections were air dried and stored at −80° C. Cryostat cut sections were dried for 45 min at RT. For antigen-retrieval the slides were submerged in preheated citrate buffer pH 6.0 (Sigma) in a water bath at 95° C. for 15 min. The slides were washed three times with PBS (5 min, RT) and blocked in 0.3% PBST with 10% NDS for 1 h at RT. Primary antibodies were diluted in 0.1% PBST with 5% NDS and incubated overnight at 4° C. The slides were washed 3 times for 10 min with PBS. Next, secondary antibodies in blocking solution were applied at a concentration of 1:500 for 2 h at RT. Slides were washed 3 times with PBS for 10 min each, whereby the first wash contained Hoechst 33342 nuclear stain (2 μg/ml,). The slides were mounted with coverslips using FluoSave (CalBiochem). Image acquisition was performed using a Leica-SP5 microscope (Leica) and LAS software (Leica) or a Zeiss Observer A1 inverted microscope (Zeiss) and Zeiss Axivision software. Further image processing and analysis was performed using the ImageJ software package 32.

Immunofluorescence for Cells

Cultured cells were rinsed with PBS before fixation with 4% PFA (10 min, RT). Subsequently, the cells were washed three times with PBS (5 min, RT, shaking). If permeabilisation was required, the cells were incubated with PBST (0.1% Triton-X-100 in PBS) for 20 min at RT. The samples were then blocked in PBS supplemented with 10% normal donkey serum (NDS). Primary antibodies (Extended data Tab. 4) were diluted in PBS with 5% NDS and incubated overnight at 4° C. in a humidified chamber. Excess antibodies were washed off with three washes in PBS (10 min, RT, shaking). The primary antibodies were then labelled with secondary antibodies (Extended data Tab. 4) diluted in PBS with 5% normal donkey serum. Again, excess antibody was washed off with three washes PBS (10 min, RT, shaking). If visualisation of nuclei was required the first wash contained 2 μg/ml Hoechst 33342 (Sigma). If coverslips were used, they were mounted onto Polysine glass slides (VWR) in a drop of Fluosave (Calbiochem) and the slides were dried for at least 3 h at RT in the dark. Images were taken with a Axio-Vision (Zeiss), Leica-SP5 (Leica) or Nikon microscope. For 96 well plate assays cells were kept in PBS after staining. Further image processing and analysis was performed using the ImageJ software package.

Comet Assay.

For comet assays of freshly isolated OPCs approximately 5000 OPCs were resuspended in 100 μl PBS and mixed with 300 μl 1% low melting point agarose (37° C.). Alternatively, when OPCs were cultured prior to the assay, the cells were detached using TrypLE 1× Select (Gibco) for 8 min at 37° C. The comet assay was then performed as described in reference 33. Briefly, OPCs were centrifuged at 300 g for 5 min. at room temperature and the cell pellet was resuspended with 100 μl PBS and then mixed with 300 μl molten low-melting point agarose pre-incubated at 37° C. The cell-agarose suspension was then applied gently onto polysine slides that were pre-treated with 1% agarose and allowed 590 to solidify at 4° C. The slides were submersed in alkaline cell lysis buffer (0.3M NaOH, 100 mM EDTA, 0.1% (w/v) N592 Lauroylsarcosine (Sigma, 61745), 1.2M NaCl in ddH2O) for 16 hours at 4° C. in the dark. The slides were then electrophoresed in alkaline electrophoresis buffer (0.03M NaOH, 2 mM EDTA, pH>12.3, pre-chilled at 4° C.) for 25 min at RT with 1V/cm, whereby cm represents the distance between the electrodes. Finally, electrophoresed and propidium iodide stained DNA was visualized using a Zeiss Axiovision Fluorescence microscope (Carl Zeiss), and 50-100 nuclei per animal were visually scored according to published protocols³⁴. Statistical significance was determined comparing respective damage categories between experimental groups by a two-tailed unpaired t-test. A significant result was assumed for p<0.05.

RNA Sequencing and Downstream Analysis

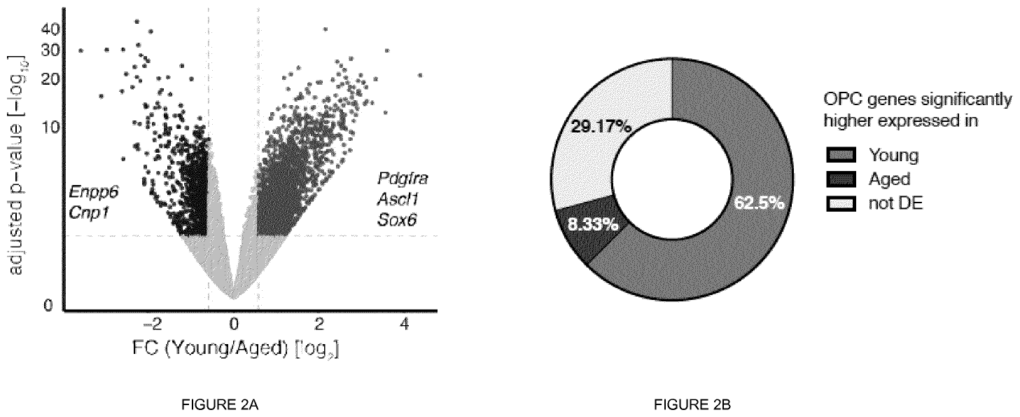

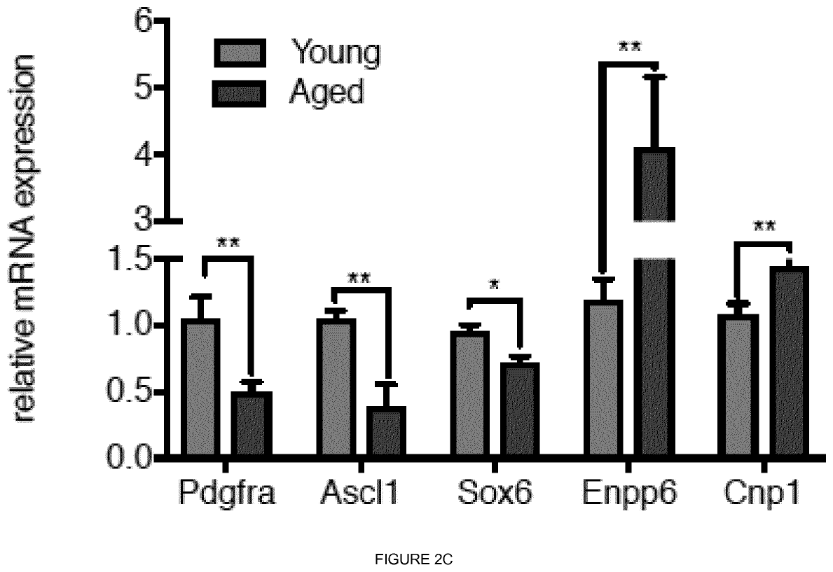

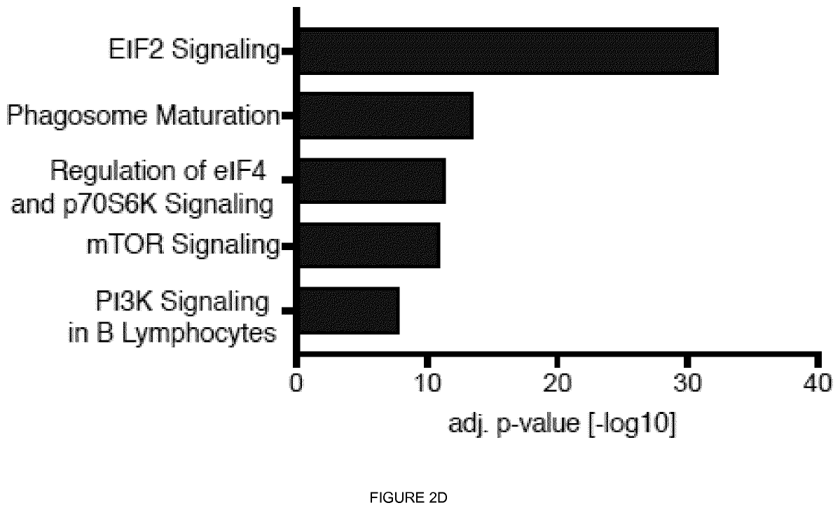

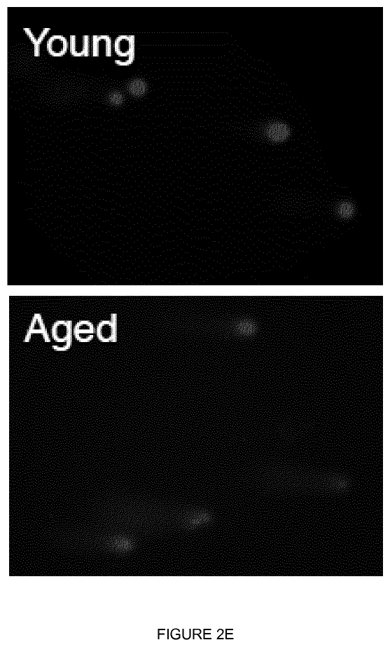

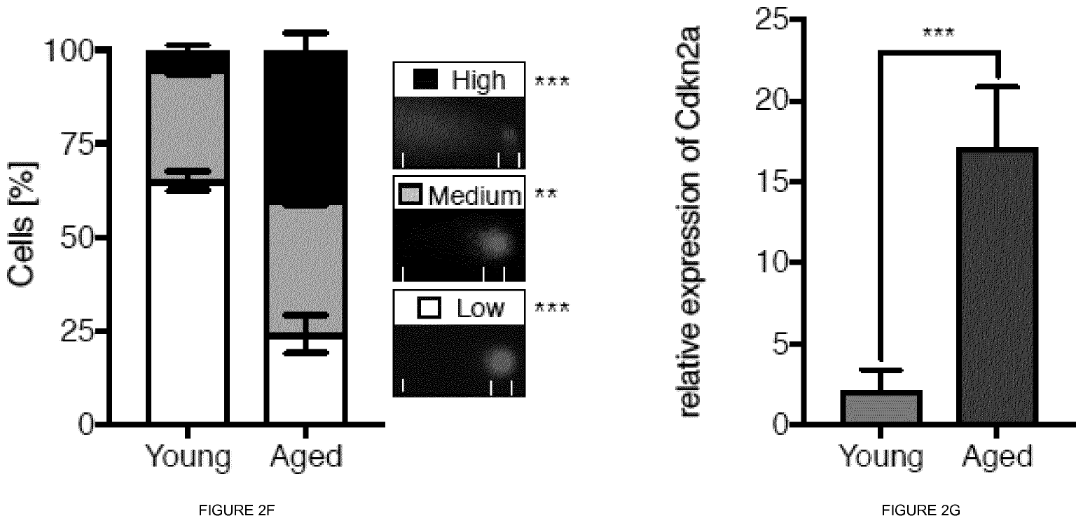

RNA was isolated from freshly purified young adult (2-3 months) and aged (20-24 months) OPCs using Qiagen RNAeasy Micro kit (Qiagen) and RNA was stored at −80° C. RNA quality was assessed by Qubit measurement and posterior RNA nanochip/picochip Bioanalyzer. Ribosomal RNA was depleted with rat-specific oligos (InDA-C technology). Sequencing libraries were prepared using 10-100 ng total RNA and the Nugen Ovation RNA-Seq Systems 1-16 for Model Organisms Kit (0349-32). Sequencing was performed on the Illumina HiSeq4000 in a pair-end 150 base pair format. Adapter sequences were removed and reads were quality-trimmed using TrimGalore. Trimmed reads were aligned to the rat reference genome (RGSC6.0/rn6) by using TopHat2³⁵ (http://ccb.jhu.edu/software/tophat, version: 2.0.13) guided by Ensembl gene models. Raw counts per gene regions were obtained by featureCounts. Replicates were evaluated, counts were normalized and differential expression of transcripts was evaluated by the R Bioconductor DESeq2 package³⁶. Expression levels were further normalized by transcript length (per kB). Transcript annotations were based on Ensembl (Release 614 82). GO term analysis as performed using the goseq package. For ingenuity pathway analysis we used differentially expressed genes with an adjusted p-value cutoff (p.adj<0.05). The OPC gene data set for FIG. 2B was taken from Table 51 from Marques et al.¹⁹.

RNA Isolation and qRT-PCR

RNA was isolated from freshly purified OPCs or from cultured OPCs according to the Directzol RNA MicroPrep Kit (Zymo Research; R2061). All RNA samples were stored at −80° C. prior to further processing. cDNA was generated using the QuantiTect Reverse Transcription Kit's according to the instructions of the manufacturer (Qiagen; 205310). For RT-qPCR, primers (see Extended Data Tab. 5) were used at a concentration of 400 μM. The efficiency of each primer was greater than −95% as determined for each primer pair by serial dilutions of OPC cDNA. cDNA, primers, and the Syber Green Master Mix (Qiagen; 04141) were mixed as instructed by the manufacturer, and RT-qPCR and melting curve analysis were performed on Life Technologies' Quantstudio 6 Flex Real-Time PCR System. Fold changes in gene expression were calculated using the delta delta Ct method in Microsoft Excel. Statistical significance was determined using two-tailed unpaired t-tests assuming equal variances.

Statistical Analysis

All statistical analysis was performed in GraphPad Prism (GraphPad Software, Inc.) or R. For data derived from the quantification of immunohistochemical staining, comparisons between two groups were performed with an unpaired t-test assuming two-tailed distribution and equal variances. In cases where more 637 than two groups were compared to each other, a one-way analysis of variance (ANOVA) was performed assuming equal variances, followed by an appropriate post-test to compare individual groups. For ranking analysis of remyelination, the non-parametric Mann-Whitney test was used to determine whether two groups differed in their extent of remyelination. A comparison of the extent of remyelination between three groups was performed using a Kruskal-Wallis test followed by Dunn's post-test to compare individual groups. For qRT PCR data two groups were compared to each other using unpaired two-tailed t-test. For data derived from comet assays damage categories would be tested between treatment groups using unpaired two-tailed t646 tests. For all statistical tests, differences were considered significant at p<0.05.

Results

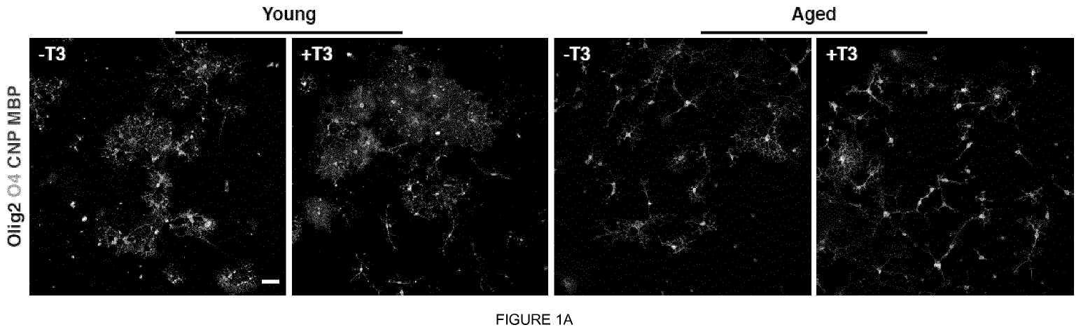

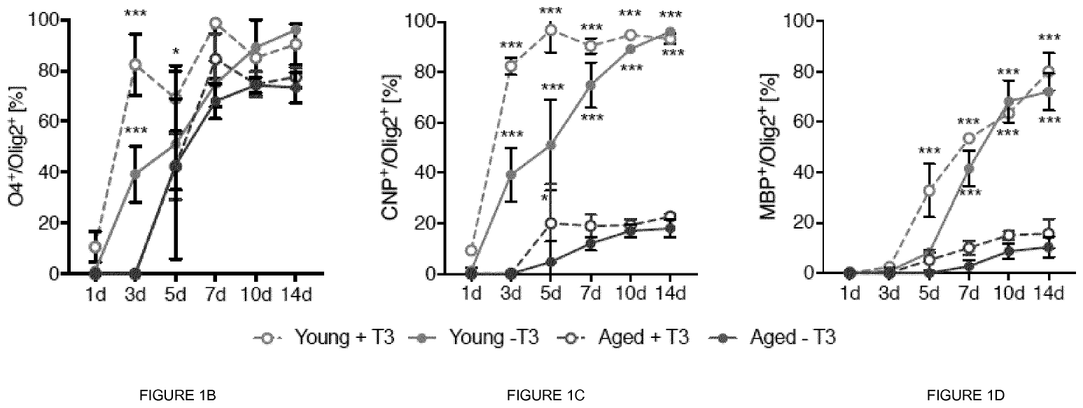

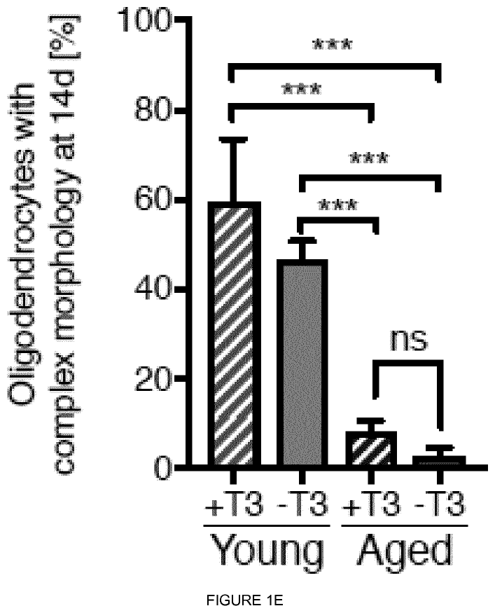

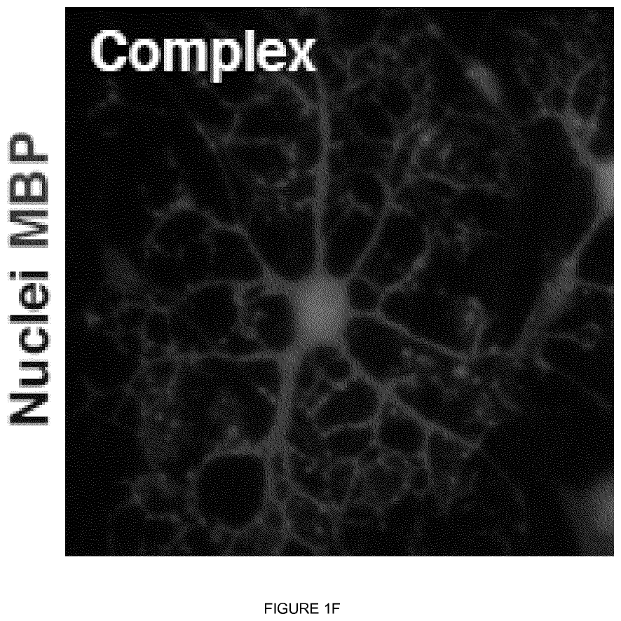

We first asked whether age-related changes in OPCs contribute to the differentiation delay observed in aged animals during remyelination⁴. Studies of OPC ageing have been hampered by the technical challenges of culturing OPCs isolated from the aged adult rodent CNS. Thus, we first optimized existing protocols¹¹ to establish cultures of adult OPCs from young adult (2-3 months) and aged (20-24 months) rats. This enabled us to compare the differentiation efficiency of OPCs isolated from the young adult and aged CNS. Cultured OPCs from young adult and aged rats increased their expression of 04 ganglioside, a marker for early differentiating oligodendrocytes, indicating that OPCs from both young adults and aged animals can commit to the adults (henceforth referred to as young OPCs) differentiated into mature CNPase+ and MBP+ oligodendrocytes, fewer than 20% of OPCs from aged adults (aged OPCs) acquired these markers within the same period (FIG. 1a, c), revealing a slower inherent rate of differentiation. We next assessed how adult OPCs responded to thyroid hormone (T3), a well-established promoter of OPC differentiation 12. While T3 accelerated the differentiation of young OPCs (FIG. 1a-d), there was no significant effect on the differentiation of aged OPCs (FIG. 1a-d). Similar results were obtained with other factors known to have pro-differentiation effects on OPCs derived from newborn animals or pluripotent stem cells, such as 9-cis-retinoic acid 13, miconazole¹⁴ and benztropine¹⁵, all of which enhanced differentiation in young (Extended Data FIG. 2b, c), but not aged OPCs. Thus, ageing OPCs become less responsive to factors that induce differentiation, which likely contributes to the failure of oligodendrocyte lineage differentiation characteristic of many non-remyelinating chronic MS lesions¹⁶.

To identify differentiation gene/pathway changes in ageing, we performed RNA-seq of OPCs isolated from young adult and aged rats. Approximately 20% of all genes were differentially expressed with Extended Data Fig. (1.5-fold change in expression, FDR<0.05, FIG. 2a). Among the genes more highly expressed in young adult OPCs were genes important for OPC self-renewal, including Pdgfra, Asci1 and Ptprz1 17. In contrast, aged OPCs expressed higher levels of the early differentiation markers Cnp1, Sirt2 and Enpp6, suggesting that aging OPCs normally exist in a more differentiated state than young cells¹⁸. To explore this further, we used a published gene dataset that contains genes specifically enriched in adult OPCs¹⁹. We found that these OPC genes were highly differentially expressed between young and aged OPCs. 62.5% of these genes were significantly higher expressed is young OPCs (FIG. 2b), results that were validated by qRT-PCR for selected genes (FIG. 2c). To identify pathways that might contribute to the aged OPC state we used ingenuity pathway analysis on genes preferentially expressed in aged OPCs, which identified mTOR signalling as one of the top 5 predicted pathways (FIG. 2d). mTOR activity is a crucial regulator of adult stem cell quiescence, activation and differentiation^{20,21} and linked to cellular aging²², where increased and dysregulated mTOR activity is associated with DNA damage and cellular senescence²³⁻²⁵. We therefore predicted that both DNA damage and markers of senescence would increase with adult OPC aging. Consistent with this prediction, comet assays revealed that aged OPCs had significantly more DNA damage than young adult OPCs (FIG. 2e, f), and expressed eight-fold higher levels of the senescence marker Cdkn2a (p16, FIG. 2f). We therefore hypothesized that dysregulation of the mTOR pathway is a significant contributor to functional decline in aging adult OPCs.

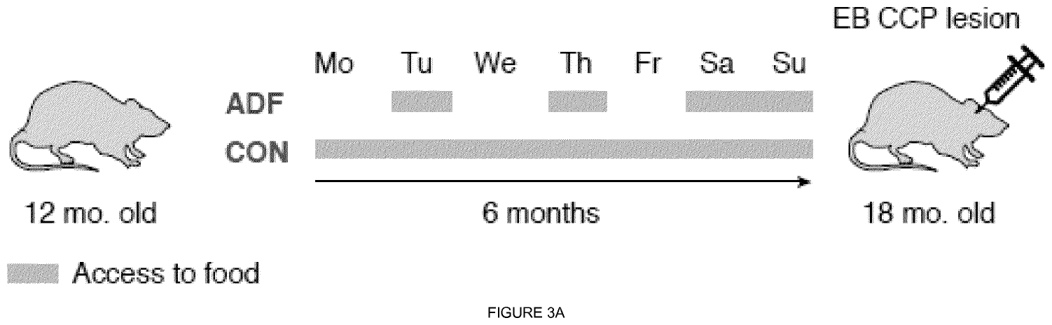

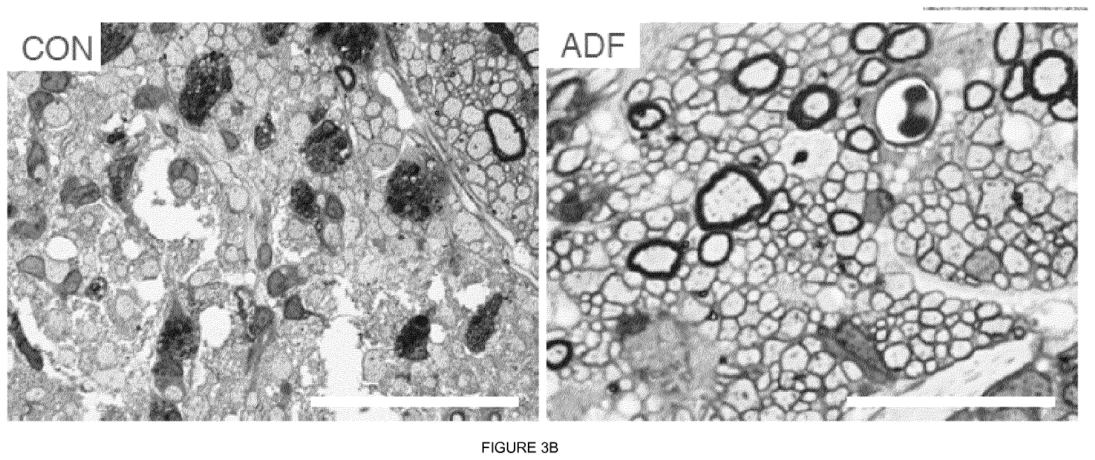

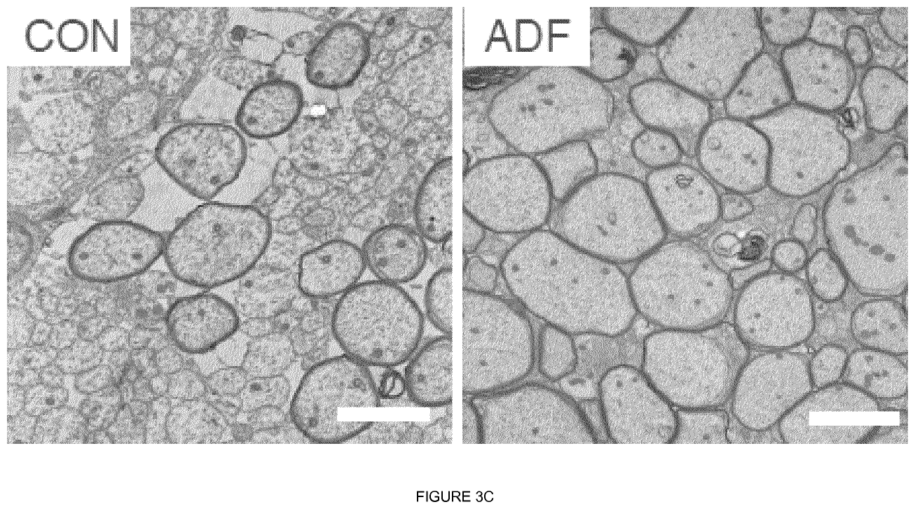

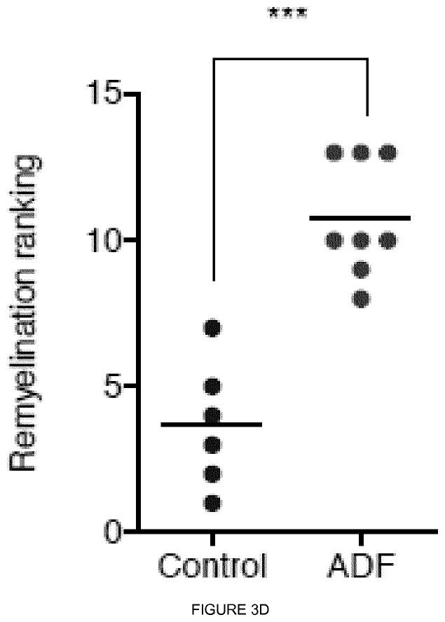

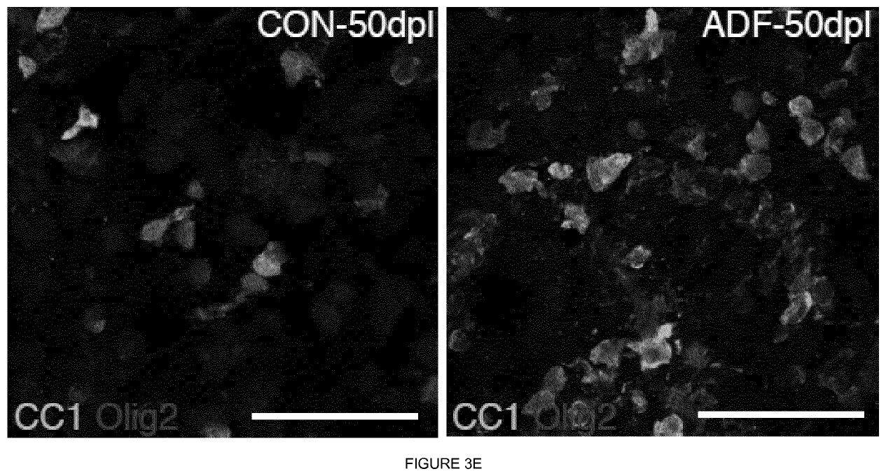

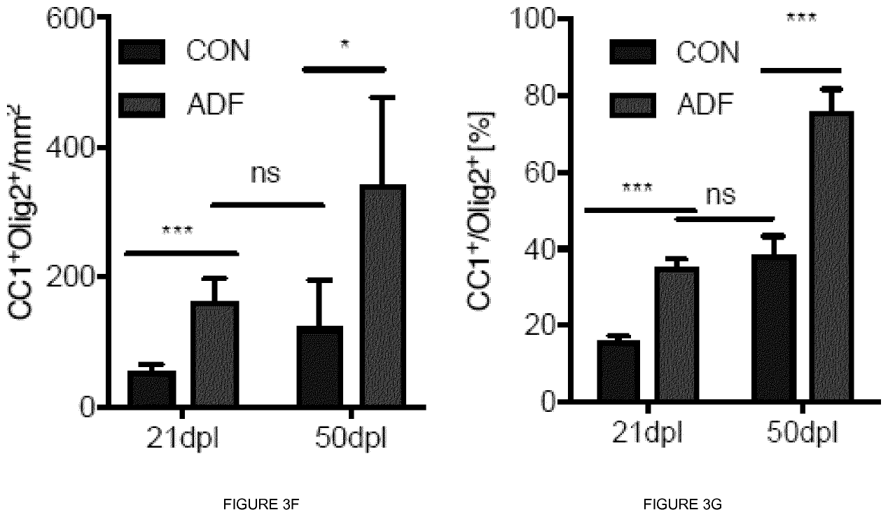

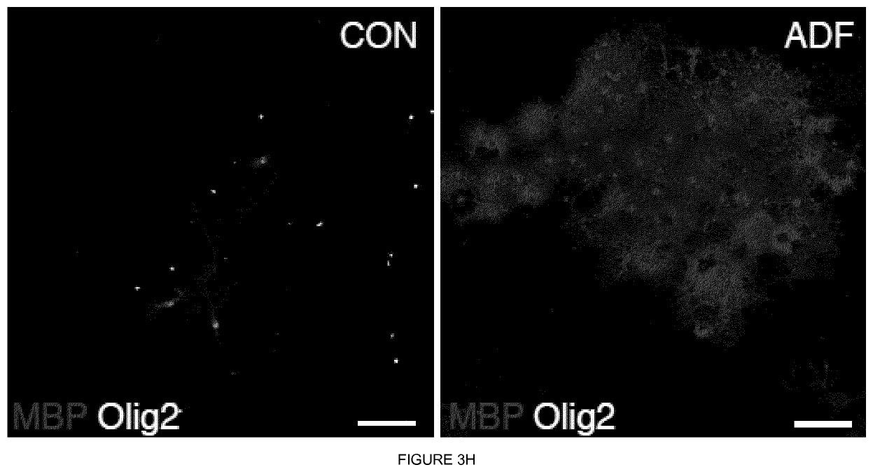

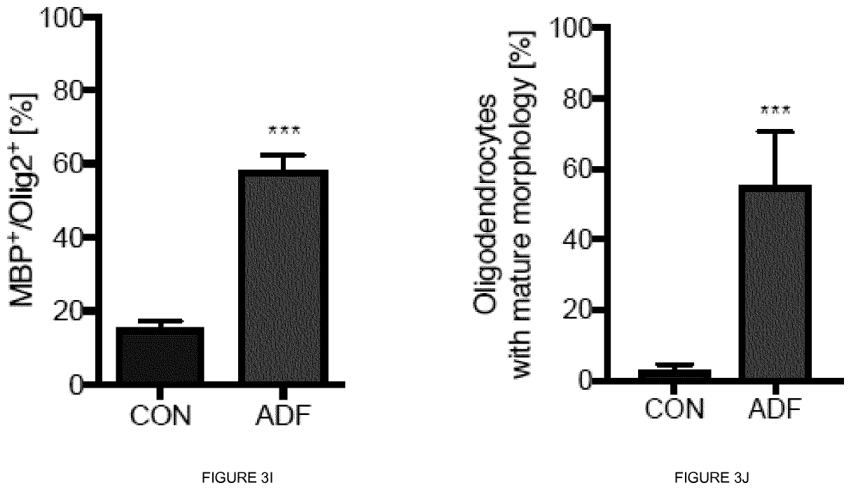

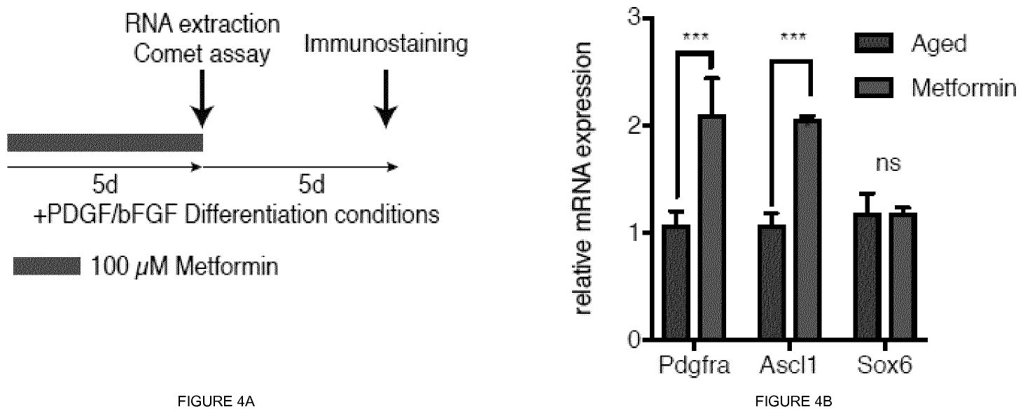

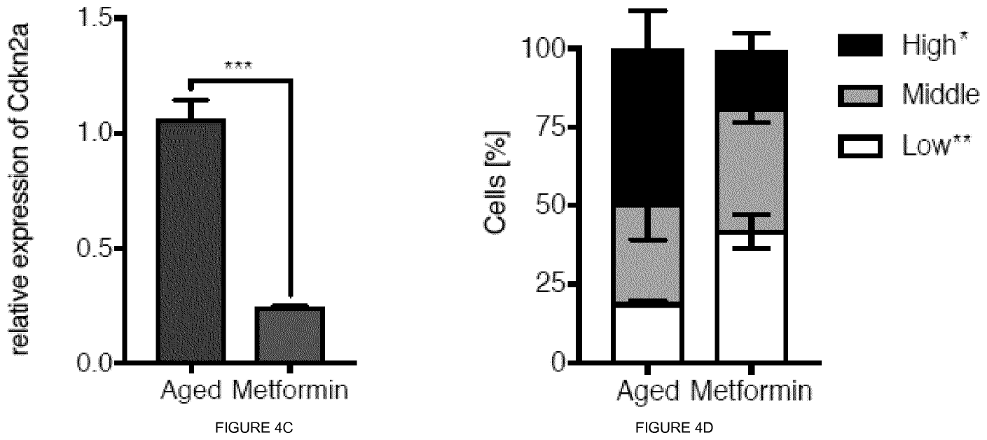

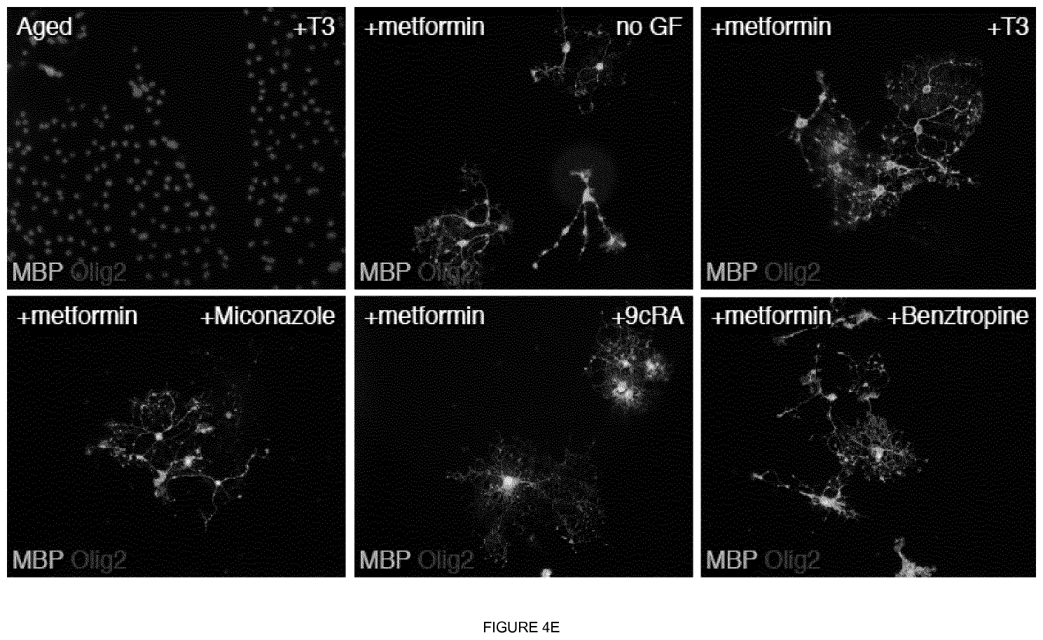

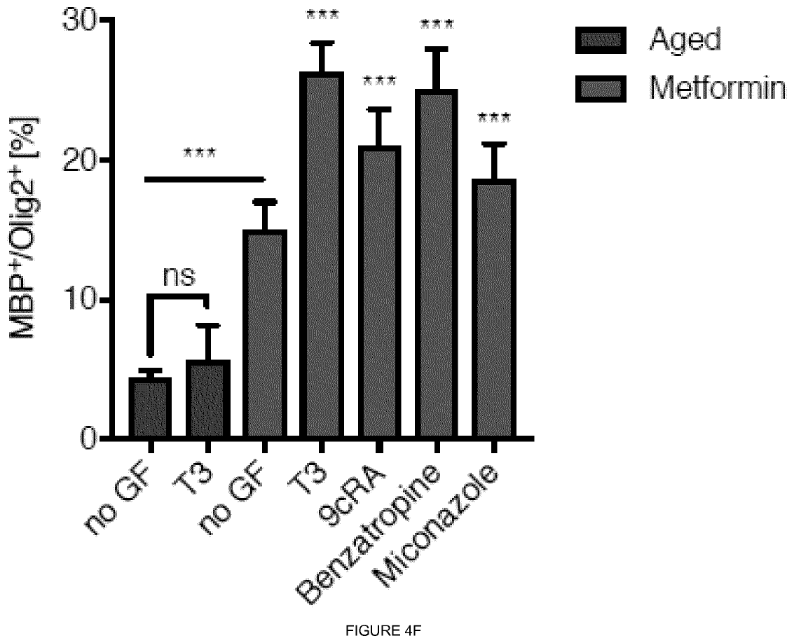

The mTOR pathway is a central intracellular nutrient signalling sensor. We therefore explored physiological strategies known to alter the effects of aging through the manipulation of nutrient signalling pathways, as a potential strategy for improving remyelination in aged animals. Dietary restriction is the most effective intervention reported to alter the aging process²⁶ and it does so in part by altering adult stem cell function²⁷. To test the effect of dietary restriction on remyelination we subjected 12 month old rats to alternate day fasting (ADF) for 6 months (FIG. 3a). At the end of this period, we induced demyelination in cerebellar white matter by the focal injection of ethidium bromide, a well-established in vivo model for studying remyelination²⁸. We assessed the degree of remyelination 50 days post lesion (dpi) in semi-thin resin sections stained with toluidine blue (FIG. 3 b, c). While remyelination in ad libitum fed animals was restricted to the border of the lesion, animals undergoing ADF consistently exhibited near complete remyelination (FIG. 3b-d). To explore ADF enhanced remyelination in aged animals further we characterised the recruitment, proliferation and differentiation of OPCs during remyelination. At 7 dpl there was no difference in the density of OPCs within the lesions of animals subjected to ADF or ad libitum feeding. In contrast, the density of mature oligodendrocytes (Olig2+/CC1+) was two-fold greater in lesions in ADF animals compared to those in controls at both 21 and 50 dpl (FIG. 3e-g). Moreover, the proportion of Olig2+ cells expressing CC1 was significantly higher in the lesions of ADF rats at both time points relative to controls, suggesting enhanced OPC differentiation (FIG. 3h). To determine if the enhanced differentiation capacity of OPCs into oligodendrocytes was due to intrinsic alterations within the OPCs, we isolated OPCs from ADF and control animals. We found that OPCs derived from ADF animals differentiated into MBP+ oligodendrocytes more rapidly than those from control rats (FIG. 3i-k), and with an efficiency comparable to OPCs from animals expressed higher levels of OPC self-renewal genes, had less DNA damage and expressed lower levels of Cdkn2a. Thus, we concluded that ADF restored remyelination in part through rejuvenation of aged OPCs. Mechanistically, dietary restriction is known to work, in part, through a reduction of mTOR signaling and an increase of signaling through AMPK pathways. mTOR inhibition using rapamycin ameliorates some of the hallmarks of aging, including DNA damage. It can also restore the differentiation potential of aged OPCs is added prior to the onset of differentiation. However, inhibition of mTOR is also a known to impede OPC differentiation²⁹, making it an unfavorable target. For this reason, we focused on the AMPK pathway, which works antagonistically to mTOR and is a key regulator of stem cell homeostasis 30. To test if activation of the AMPK pathway can restore the function of aged OPCs, we exposed OPCs cultured from aged rats to metformin. First, we used qRT-PCR to assess if metformin treatment was sufficient to increase the expression of OPC self-renewal. Metformin treated cells increased their expression of Pdgfra and Ascl1 (FIG. 4b), had less DNA damage, as indicated by comet assays (FIG. 4d), and expressed significantly less Cdkn2a (FIG. 4c), suggesting that metformin is sufficient to phenocopy at least some of the effects of ADF.

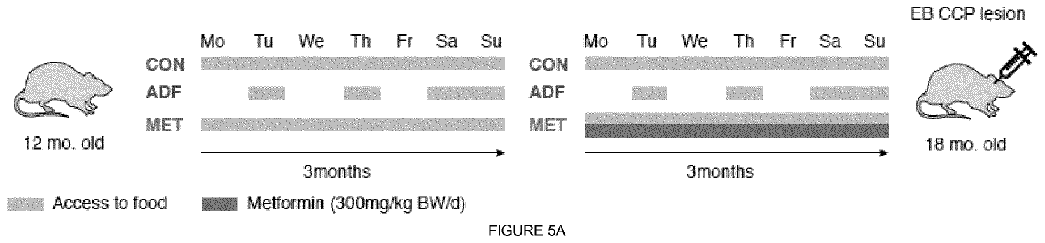

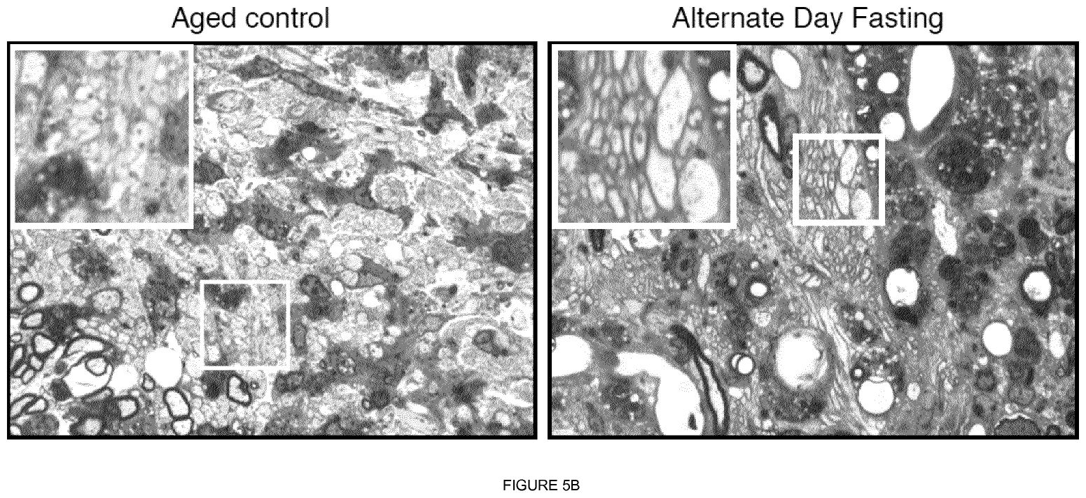

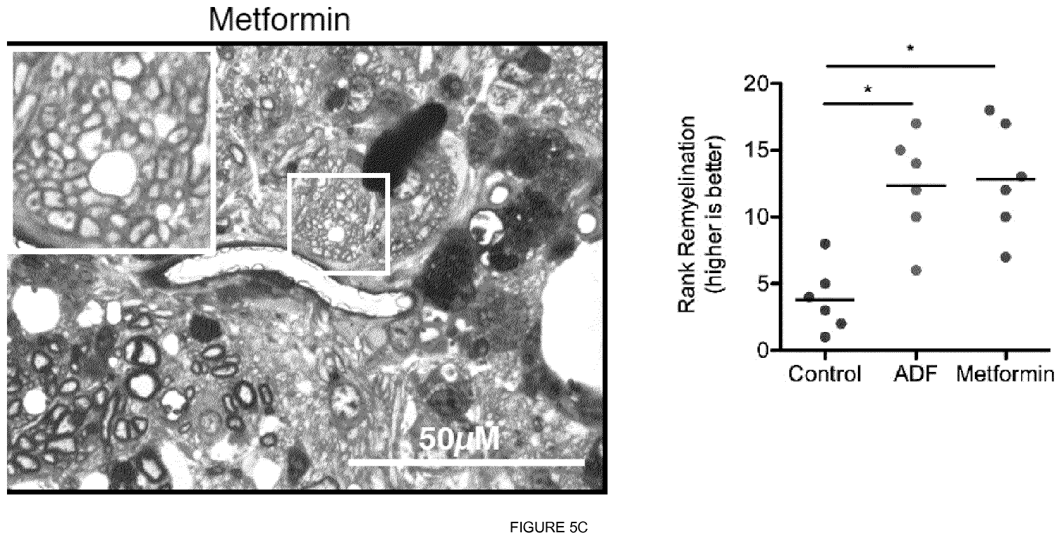

Next, we asked if exposure to metformin was also sufficient to restore the differentiation potential of aged OPCs and their responsiveness to differentiation factors. We found that metformin enhanced differentiation of aged OPCs when added prior to the addition of pro-differentiation compounds. Further, we observed a significant increase in the proportion of MBP expressing oligodendrocytes with highly arborized morphology in the presence of pro-differentiation factors. Finally, we tested if metformin treatment could mimic ADF. Aged rats, fed ad libitum, received metformin in their drinking water for three months prior to lesion induction (FIG. 5a). Metformin treated animals exhibited a degree of remyelination that was comparable to animals undergoing ADF and that was significantly higher compared to control animals (FIG. 5b, 5c).

Understanding how aging affects remyelination and how these effects may be reversed is critical to devising regenerative interventions with which to combat progressive MS. Our results reveal that OPCs lose their capacity to respond to pro-differentiation factors with increasing age. This is likely to pose significant constraints on the effectiveness of remyelination therapies based on enhancement of differentiation. Importantly, using fasting and metformin to ameliorate hallmarks of aging we show that the age dependent loss of OPC potential is reversible, which is a pre-requisite for pharmacological interventions targeting endogenous OPCs to stimulate remyelination. Understanding and overcoming the effects of aging on OPCs will be equally important as the modulation of the extrinsic factors to generate a permissive environment for regeneration to successfully implement future remyelination therapies. Interventions, such as dietary restriction or drugs that mimic its effects are likely therefore to play a significant role in the development of effective therapies to promote remyelination.

### REFERENCES

1. Franklin, R. J. M. & ffrench-Constant, C. Nature Rev. Neurosci. 9, 839-855 (2008).
2. Moyon, S. et al. J. Neurosci. 35, 4-20 (2015).
3. Zawadzka, M. et al Cell Stem Cell 6, 578-590 (2010).
4. Sim, F. J. et al J. Neurosci. 22 2451-2459 (2002).
5. Oh, J., Lee, Y. D. & Wagers, A. J. Nature Med. 20, 870-880 (2014).
6. Franklin, R. J. M. et al Nature Rev. Neurol. 8, 624-634 (2012).
7. Shen, S. et al. Nature Neurosci. 11, 1024-1034 (2008).
8. Hinks, G. L. & Franklin, R. J. Mol. Cell. Neurosci. 16, 542-556 (2000).
9. Natrajan, M. S. et al. Brain 138, 3581-3597 (2015).
10. Ruckh, J. M. et al. Cell Stem Cell 10, 96-103 (2012).
11. Dugas, J. C. & Emery, B. Cold Spring Harb. Protoc. 2013, 810-814 (2013).
12. Gao F. B., Apperly, J. & Raff, M. Dev. Biol. 197, 54-66 (1998).
13. Huang, J. K. et al. Nature Neurosci. 14, 45-53 (2010).
14. Najm, F. J. et al. Nature 522, 216-220 (2015).
15. Deshmukh, V. A. et al. Nature 502, 327-332 (2013).
16. Kuhlmann, T. et al. Brain 131, 1749-1758 (2008).
17. Emery, B. Science 330, 779-782 (2010).
18. Liu, L. et al. Cell Reports 4, 189-204 (2013).
19. Marques, S. et al. Science 352, 1326-1329 (2016).
20. Rodgers, J. T. et al. Nature 510, 393-396 (2014).
21. Mihaylova, M. M., Sabatini, D. M. & Yilmaz, O. H. Cell Stem Cell 14, 292-305 (2014).
22. Laplante, M. & Sabatini, D. M. Cell 149, 274-293 (2012).
23. Yilmaz, 0. H. et al. Nature 441, 475-482 (2006).
24. Castilho, R. M. et al Cell Stem Cell 5, 279-289 (2009).
25. Chen, C., Liu, Y., Liu, Y. & Zheng, P. Science 2, ra75-ra75 (2009).
26. Fontana, L. & Partridge, L. Cell 161, 106-118 (2015).
27. Cerletti, M et al. Cell Stem Cell 10, 515-519 (2012).
28. Woodruff, R. H. & Franklin, R. J. Glia 25, 216-228 (1999).
29. Tyler, W. A. et al. J. Neurosci. 29, 6367-6378 (2009).
30. Igarashi, M. & Guarente, L. Cell 166, 436-450 (2016).
31. Arnett, H. A. et al. Science 306, 2111-2115 (2004).
32. Schindelin, J. et al Nature Meth. 9, 676-682 (2012).
33. Olive, P. L. & Banath, J. P. Nature Protocols 1, 23-29 (2006).
34. Collins, A. R. Mol. Biotechnol. 26, 249-261 (2004).
35. Kim, D. et al. Genome Biol. 14, R36 (2013).
36. Love, M. I., Huber, W. & Anders, S. Genome Biol. 15, 550 (2014).

1. A therapeutic combination comprising an AMPK agonist and a differentiation factor.

2. A method of treating a demyelinating disease comprising administering a therapeutic combination according to claim 1 to an individual in need thereof.

3. A therapeutic combination according to claim 1 for use in a method of treatment of a demyelinating disease.

4. Use of a therapeutic combination according to claim 1 in the manufacture of a medicament for use in the treatment of a demyelinating disease.

5. A method, combination for use or use of any one of claims 2 to 4 wherein the demyelinating disease is multiple sclerosis.

6. A method, combination for use or use of claim 5 wherein the multiple sclerosis is progressive multiple sclerosis.

7. A combination, method, combination for use or use of any one of the preceding claims wherein the therapeutic combination promotes remyelination of demyelinated neurons.

8. A combination, method, combination for use or use of any one of the preceding claims wherein the therapeutic combination reduces or inhibits the degeneration of demyelinated neurons.

9. A combination, method, combination for use or use of any one of the preceding claims wherein the AMPK agonist is metformin, buformin, phenformin, 5-aminoimidazole-4-carboxamide ribonucleotide (AICAR), 2-deoxy-D-glucose (2DG), salicylate or A-769662 (6,7-Dihydro-4-hydroxy-3-(2′-hydroxy[1,1′-biphenyl]-4-yl)-6-oxo-thieno[2,3-b]pyridine-5-carbonitrile).

10. A combination, method, combination for use or use of any one of the preceding claims wherein the AMPK agonist is metformin.

11. A combination, method, combination for use or use of any one of the preceding claims wherein the differentiation factor promotes the differentiation of oligodendrocyte progenitor cells (OPCs) into oligodendrocytes.

12. A combination, method, combination for use or use of any one of the preceding claims wherein the differentiation factor is a retinoid X receptor (RXR) agonist

13. A combination, method, combination for use or use of claim 12 wherein the retinoid X receptor (RXR) agonist is bexarotene

14. A combination, method, combination for use or use of any one claims 1 to 11 wherein the differentiation factor is a selective histamine Hi antagonist.

15. A combination, method, combination for use or use of claim 14 wherein the selective histamine Hi antagonist is clemastine.

16. A combination, method, combination for use or use of any one claims 1 to 11 wherein the differentiation factor is a selective M1 muscarinic acetylcholine receptor antagonist.

17. A combination, method, combination for use or use of claim 16 wherein the M1 muscarinic acetylcholine receptor antagonist is benzatropine.

18. A combination, method, combination for use or use of any one claims 1 to 11 wherein the differentiation factor is a glucocorticoid receptor antagonist.

19. A combination, method, combination for use or use of claim 18 wherein the glucocorticoid receptor antagonist is miconazole.

20. A combination, method, combination for use or use of any one claims 1 to 11 wherein the differentiation factor is a thyroid hormone receptor agonist.

21. A combination, method, combination for use or use of claim 20 wherein the thyroid hormone receptor agonist is thyroid hormone T3 or T4.

22. A pharmaceutical composition comprising a therapeutic combination according to any one of claims 1 and 7 to 21.

23. A therapeutic combination according to any one of claims 1 and 7 to 21 for use in a method of treatment of the human or animal body.
